(19) United States
(12) Patent Application Publication
(10) Pub. No.: US 2023/0290489 A1
(43) Pub. Date: Sep. 14, 2023

(54) INVENTORY PREDICTION MANAGEMENT SYSTEM

(71) Applicant: GrindFoundry, Inc., San Jose, CA (US)

(72) Inventors: Jonathan Rat, San Jose, CA (US); Benjamin Kolin, Sunnyvale, CA (US); Nimish Sheth, Sunnyvale, CA (US); Christine Liu, San Jose, CA (US)

(73) Assignee: GrindFoundry, Inc., San Jose, CA (US)

(21) Appl. No.: 17/690,857

(22) Filed: Mar. 9, 2022

(51) Int. Cl. G16H 40/20; G16H 70/20; G16H 50/20; G06N 5/04; G06N 20/00

(52) U.S. Cl. CPC G16H 40/20; G16H 70/20; G16H 50/20; G06N 5/04; G06N 20/00; G06Q 40/08

(57) ABSTRACT

Method, systems, and apparatus for receiving appointment data about one or more appointments, wherein the appointment data comprises one or more procedural codes for each of the one or more appointments; generating an estimated amount of inventory used for each of the one or more appointments based at least on the one or more procedural codes; calculating a remaining amount of available inventory from the estimated amount of inventory used; generating a threshold for each supply in the remaining amount of available inventory based at least on historical purchase data for the supply, wherein the historical purchase data comprises a duration from order to arrival; determining, for a given supply, that the remaining amount of available inventory for the given supply is under the generated threshold; and in response to the determining, generating a notification for a user to purchase the given supply.

### TECHNICAL FIELD

This disclosure relates to a system that enables service providers to manage appointments, scheduling, inventory, and claims.

### SUMMARY

Traditional patient management systems used by service providers like dental or medical practices manage patient contact information and statuses for each patient such as whether they are a prospective patient or current patient. These management systems also can track history of procedures performed on the patients by the service provider.

The patient management system described herein generates candidate appointment slots from appointment constraints. Appointment constraints are conditions that should be satisfied by each candidate appointment slot. An appointment constraint, for example, can be that the candidate appointment slot should have a particular duration. Other appointment constraints include ensuring other providers, e.g., dentists, have open calendar availability. By considering calendar availability from multiple providers as well as multiple procedural codes handled by the providers, the patient management system generates candidate appointment slots. This allows an operator of the patient management system to quickly identify optimal candidate appointment slots for patient appointments.

The patient management system recommends procedures for scheduling. The patient management system determines a recommendation score for candidate patient procedures using a machine-learning model. The recommendation score can also be based on insurance provider data for the patient as well as provider data. The patient management system recommends procedures for scheduling with patients based on the recommendation score. This allows a practice to, using the patient management system, generate more revenue, identify lower risk procedures to perform, or identify procedures to perform that are preferential to providers.

The patient management system manages inventory using a prediction management system. The prediction management system tracks available inventory as well as appointments that have occurred or procedures that have been performed to generate an estimate of inventory used based on procedural codes associated with the appointments. The prediction management system determines a threshold under which the prediction management system sends a notification to purchase more inventory for the practice. The prediction management system ensures the practice will have inventory to conduct procedures, thereby maintaining continuous operation of the practice. This also prevents the practice from holding excess inventory, which improves cash flow.

The patient management system recommends claim filings for the practice to submit to claim processors managed by insurance providers. Claim filings include at least one or more procedural codes and an amount billed. The patient management system can generate a recommendation to change the one or more procedural codes in the claim filing based on a machine-learning model. This improves accuracy of tracking the amount that will be received from the insurance provider for budgeting purposes. The patient management system also can recommend appealing a rejected claim filing from the claim processors. This may improve revenue for the practice by capturing a maximal amount due from the insurance provider.

In one aspect, a method comprising, by one or more computing devices: receiving a request for appointment availability, wherein the request for appointment availability comprises one or more procedural codes; determining an aggregate appointment duration and provider type data based at least on the one or more procedural codes from a first mapping of procedural codes to procedural durations and a second mapping of procedural codes to provider type data; identifying a plurality of appointment constraints based at least on the request for appointment availability, the aggregate appointment duration, and the provider type data; and applying the plurality of appointment constraints to calendar availability data of a plurality of providers to generate a plurality of candidate appointment slots based at least on the plurality of appointment constraints.

The request for appointment availability comprises preference data for one or more time slots and one or more providers. The request for appointment availability comprises preference data for one or more rooms. The request for appointment availability comprises first patient data and second patient data, wherein the first patient data is associated with a first plurality of procedural codes and the second patient data is associated with a second plurality of procedural codes. The request for appointment availability comprises preference data indicating whether the plurality of candidate appointment slots should overlap in time or should be contiguous in time. Generating the plurality of appointment constraints is further based at least on room availability, inventory availability, office opening hours, provider working hours, provider holidays, patient to provider preference data, provider to room preference data, or provider double-booking preferences. Generating a plurality of candidate appointment slots comprises: identifying a plurality of providers with different provider type data based at least on the second mapping of procedural codes to provider type data; assigning a time slot to each provider in the plurality of providers based at least on the first mapping of procedural codes to procedural durations; and generating a candidate appointment slot from the plurality of time slots. Generating data indicating lost revenue from a third mapping of procedural codes to revenue, wherein the plurality of candidate appointment slots comprises time in the past.

In another aspect, a method comprising, by one or more computing devices: generating a plurality of candidate patient procedures based at least on one or more procedural codes; determining, for each of the plurality of candidate patient procedures, a risk score for the candidate patient procedure using a machine-learning model trained on data comprising one or more procedure types, patient information, and frequency scores of procedural incidents for each procedure type, and severity scores of procedural incidents for each procedure type; determining, for each of the plurality of candidate patient procedures, a recommendation score for the candidate patient procedure based at least on insurance provider data for the patient in the candidate patient procedure, a mapping of the candidate patient procedure to revenue per procedure, and the risk score for the candidate patient procedure; and generating one or more appointment creation messages for distribution to a plurality of candidate patients based on the recommendation scores.

The insurance provider data further comprises a mapping of insurance provider to covered procedures and costs paid per covered procedure. The recommendation score is further based at least on provider data comprising a mapping of insurance provider to service provider covered procedures and costs paid per covered procedure. Prior to generating the one or more appointment creation messages: generating one or more appointment creation suggestions based on the recommendation scores; and in response to receiving an approval of one or more of the appointment creation suggestions, generating the one or more appointment creation messages. Generating the one or more appointment creation messages comprises: identifying a plurality of appointment constraints based on the plurality of candidate patient procedures; and applying the plurality of appointment constraints to calendar availability data to generate a plurality of candidate appointment slots for one or more providers based at least on the plurality of appointment constraints, wherein the one or more appointment creation messages comprise information about the candidate patient procedures and one or more of the plurality of candidate appointment slots for the one or more providers. The plurality of appointment constraints comprises preference data from one or more providers.

In another aspect, a method comprising, by one or more computing devices: receiving appointment data about one or more appointments, wherein the appointment data comprises one or more procedural codes for each of the one or more appointments; generating an estimated amount of inventory used for each of the one or more appointments based at least on the one or more procedural codes; calculating a remaining amount of available inventory from the estimated amount of inventory used; generating a threshold for each supply in the remaining amount of available inventory based at least on historical purchase data for the supply, wherein the historical purchase data comprises a duration from order to arrival; determining, for a given supply, that the remaining amount of available inventory for the given supply is under the generated threshold; and in response to the determining, generating a notification for a user to purchase the given supply. Generating the threshold for each supply comprises: training a machine learning model on data comprising inventory data, date data, historical appointment data, and the historical purchase data; inferring, using the trained machine learning model, the generated threshold. Generating the threshold is based at least on a rate of consumption of each supply. Receiving an updated inventory amount; and adjusting the remaining amount based at least on the updated inventory amount. Assigning the notification a priority based on at least a frequency of procedural codes in the appointment data. Assigning the notification a priority based on at least revenue score or a risk score.

In another aspect, training a machine-learning model on data comprising a plurality of originally filed procedural codes, a plurality of revised procedural codes, one or more insurance providers, descriptions from procedures associated with the procedural codes, procedural approval statuses, and a plurality of fee amounts; receiving one or more candidate procedural codes with respective fee amounts; and using the machine-learning model to generate one or more recommended procedural codes that are different from the one or more candidate procedural codes. The machine-learning model is further trained on a plurality of date data associated with the originally filed procedural codes. The descriptions from procedures associated with the procedural codes are represented as embeddings. Providing the generated one or more recommended procedural codes for display on a user interface. Replacing the one or more candidate procedural codes with the one or more recommended procedural codes; and submitting the claim filing to the claim processor. Submitting a claim filing comprising the one or more candidate procedural codes; receiving a rejected claim filing comprising the one or more candidate procedural codes; training an additional machine-learning model on appeal data comprising insurance provider data, historical claim filing data comprising first fee data and first procedural code data, historical claim rejection data comprising second fee data and second procedural code data, and historical claim appeal data comprising final fee data and final procedural code data; and using the additional machine-learning model to generate a recommendation to appeal the rejected claim filing.

### BRIEF DESCRIPTION OF THE DRAWINGS

FIG. 1 is a schematic illustration of an example appointment management architecture.

FIG. 2 is a flow chart of an example process of generating candidate appointment slots.

FIG. 3 is a flow chart of an example process of generating candidate appointment slots based on provider data.

FIG. 4 is an illustration of an example user interface showing data representing procedural codes and associated appointment durations.

FIG. 5 is an illustration of an example user interface showing generation of candidate appointment slots.

FIG. 6 is a schematic illustration of an example appointment recommendation architecture.

FIG. 7 is a flow chart of an example process of generating appointment creation messages based on recommendation scores.

FIGS. 8A-B are illustrations of example user interfaces showing generation and viewing of appointment creation messages.

FIG. 9 is a schematic illustration of an example inventory management architecture.

FIG. 10 is a flow chart of generating a notification for a user to purchase supply for more inventory.

FIG. 11 is an illustration of an example user interface showing a notification to purchase inventory based on available inventory.

FIG. 12 is a schematic illustration of an example claim filing management architecture.

FIG. 13 is a flow chart of an example process of generating recommended procedural codes for claim filings.

FIG. 14 is a flow chart of an example process of generating a recommendation to appeal rejected claims.

FIG. 15 is an illustration of an example user interface showing recommended procedural codes for claim filings.

Like reference numbers and designations in the various drawings indicate like elements.

### DETAILED DESCRIPTION

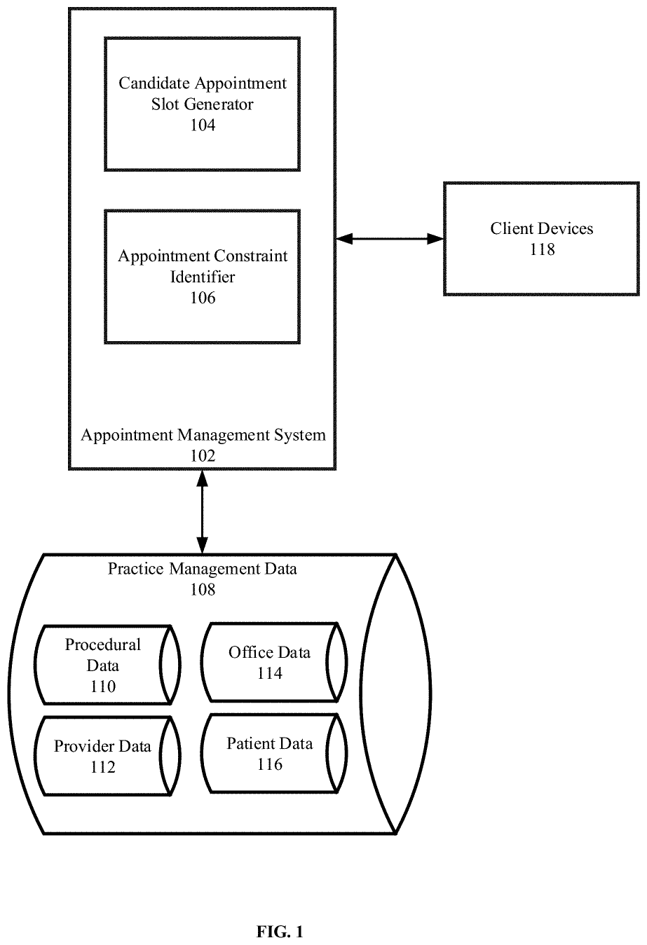

FIG. 1 is a schematic illustration of an example appointment management architecture. The appointment management architecture includes an appointment management system 102 that interfaces with practice management data 108 and client devices 118, e.g., over the Internet. Client devices can be mobile devices, desktops, or laptop computers. The client devices can be operated by employees of a health practice, for example, a dental practice. The client devices can also be operated by customers or patients of the health practice. In some implementations, the appointment management system 102 is a software-as-a-service (SaaS) based practice management platform.

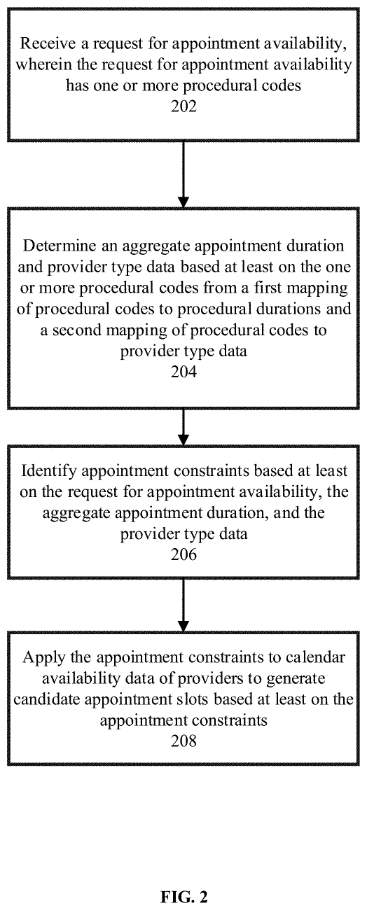

The appointment management system 102 includes a candidate appointment slot generator 104 and an appointment constraint identifier 106. The candidate appointment slot generator 104 processes practice management data 108 to generate candidate appointment slots that are provided to the client devices 118. The appointment constraint identifier 106 processes practice management data 108 and provides appointment constraints to the candidate appointment slot generator 104, which uses the appointment constraints to generate the candidate appointment slots. Generating the candidate appointment slots will be described further below in reference to FIGS. 2-5.

The practice management data 108 includes procedural data 110, office data 114, provider data 112, and patient data 116. The practice management data data 108 can be stored in a database or other forms of storage.

The procedural data 110 can include data associated with procedures, e.g., health or dental procedures. In some implementations, each procedure is associated with a procedural code, a procedure name, a simple name, an appointment duration, an overlap before time, an overlap after time, and notes inputted by providers performing the procedure. The procedural code can be unique to each procedure and represents a shorthand representation for the procedure. The overlap before time is a duration at the start of the appointment duration during which the procedure can overlap with another procedure without causing a calendar conflict. The overlap after time is a duration at the end of the appointment duration during which the procedure can overlap with another procedure without causing a calendar conflict.

The office data 114 can include data regarding operating hours and days of the practice, preferred operating hours of the practice, calendar availability for the practice, a number of available rooms, an amount of specialty equipment, and inventory amount, such as how many chairs are available, X-ray devices, intraoral devices, dental cone beam computed tomography equipment (CBCT), a room dedicated for X-rays or a specialist, or a room dedicated to adults or kids.

The provider data 112 can include data about the providers of the practice such as their names, contact information including phone number and email, operating schedules including days and hours, preferred operating schedules, procedures able to be performed and associated procedural codes, preferred procedures to perform and associated preferred procedural codes, a provider type, calendar availability data for each provider, or provider preferences regarding which patients to see or not see. A provider type can represent a specialty for the provider type, e.g., periodontist. Certain providers having a provider type can perform procedures categorized for a given specialty while other providers not having that provider type are not able to perform procedures for that given specialty. Preferred procedures by provider are also stored in the provider data 112. They are set by the provider and can represent a subset of procedures they are able to perform. For example, a provider named Doctor Black can perform procedural codes D1, D2, D3, and S1 while a provider named Dr. Pratt can perform procedural codes D1 and D2. Doctor Black can also store, in the provider data, that she prefers to perform procedural codes D3.

The patient data 116 includes data about the patients of the practice. This data includes a chart for each patient. The chart includes basic medical history, interactions with the practice and other practices, progress notes of previous treatments, descriptions of services provided, treating clinicians' identities, dates of performed procedures, radiographs, methods and materials of any anesthetics, all recommendations provided, and projected future treatments. This data can also include patient contact information, billing information, patient preferences for procedure types such as preferred rooms or chairs for procedures or preferred types of equipment used, preferred providers, preferred lengths of appointment times and dates, a mapping to other family members who are also patients of the practice, prescription data, or previous medical or dental history of the patient from other providers.

FIG. 2 is a flow chart of an example process of generating candidate appointment slots. The process can be executed by an appointment management system, e.g., using an appointment management system 102 referenced in FIG. 1.

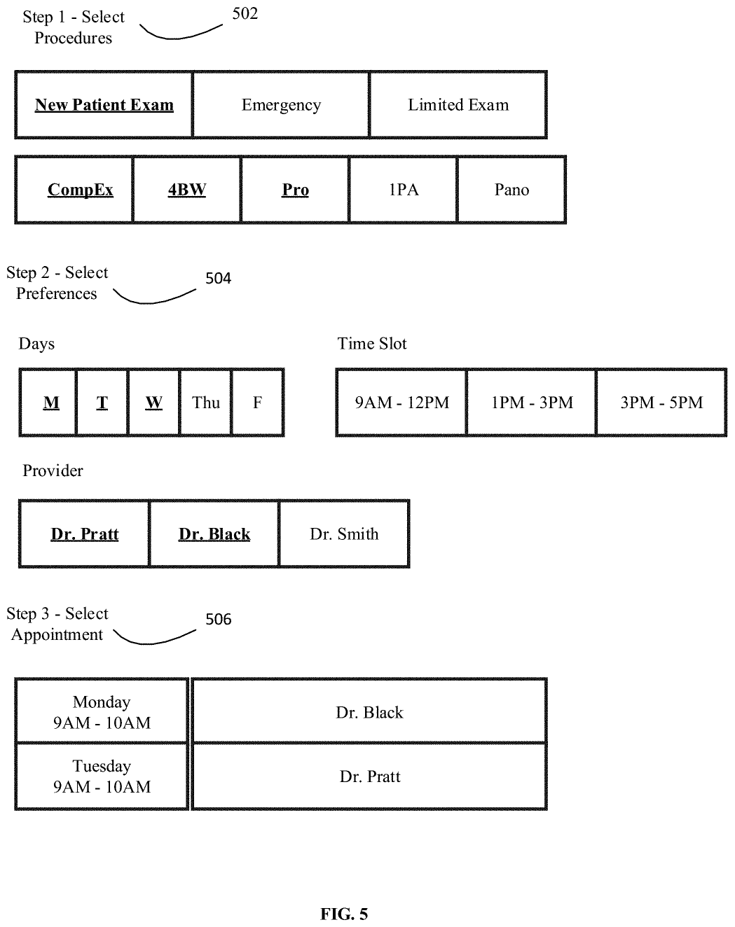

The appointment management system receives a request for appointment availability (step 202). An example user interface showing the request will be described further below in reference to FIG. 5. The request can come from a client device, e.g., client devices 118 referenced in FIG. 1. The client device can be a device operated by a health practice or a device owned by a patient of the health practice. The request for appointment availability includes one or more procedural codes. In some implementations, the procedural codes are required before candidate appointment slots are provided by the appointment management system.

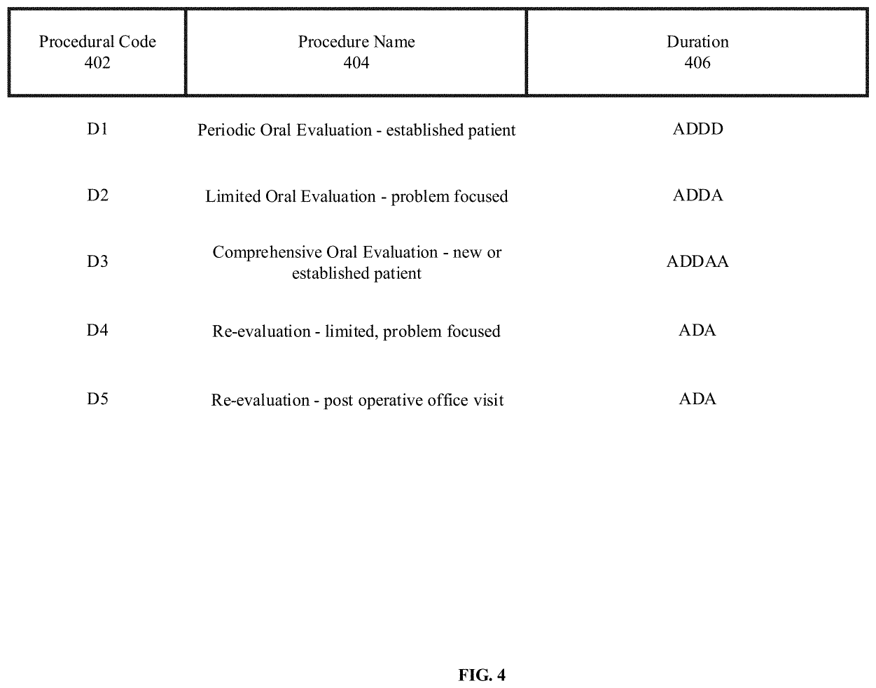

Procedural codes reference health services. They can be categorized according to service: diagnostic, preventative, restorative, or other adjunctive general services. In some implementations, the procedural codes reference the Code on Dental Procedures and Nomenclature (CDT codes), and include dental-specific categories such as endodontics, periodontics, removable prosthodontics, maxillofacial prosthetics, implant services, fixed prosthodontics, oral and maxillofacial surgery, and orthodontics. Example procedural codes will be described further below in reference to FIG. 4.

In some implementations, the request for appointment availability includes preference data. The preference data can be provided by the device owned by the practice, for example, a patient relays preferences to an operator who inputs the preference data into the device, or the preference data is inputted directly by the patient for transfer to the appointment management system.

The preference data can include preferences for certain time slots or for certain providers. The preference data for time slots can represent a variety of ranges such as any time during a given date, a time range, both a time range and a date range, or one or more days of the week in addition to a time range. The preference data for certain providers can represent a preference for a patient to be operated on by a manually chosen subset of providers working at the practice.

In some implementations, the request for appointment availability includes preference data for one or more rooms in the practice. The appointment management system stores office data, e.g., office data 114 referenced in FIG. 1. The office data includes a number of rooms in which providers can operate procedures. The preference data for certain rooms represents a patient's preference to be in a familiar environment when being operated on by a provider.

In some implementations, the request for appointment availability includes patient data from multiple patients. A patient occasionally wants to schedule an appointment for members of his or her family during the same visit. The request can include a first set of procedural codes for a first patient and a second set of procedural codes for the second patient.

In some implementations, the appointment management system receives an indication requesting to schedule an appointment for both patients and a procedural code for the first patient. The appointment management system can then retrieve a chart for the second patient that indicates projected future treatment plans. From the chart, the appointment management system can retrieve associated procedural codes for a future procedure in the projected future treatment plans and use the procedural codes to generate candidate appointment slots, which will be described further below.

In some implementations, the request for appointment availability includes preference data indicating whether candidate appointment slots should overlap in time or should be contiguous in time. In particular, a patient wanting to schedule an appointment for members of his or her family can have the appointment slots overlap in time or be sequential or contiguous in time. For example, a patient can have all three children be scheduled for a procedure from 1-2 pm, or the patient can have all three children scheduled from 1-4 pm; one child can be scheduled for 1-2 pm, another child can be scheduled for 2-3 pm, and the last child can be scheduled for 3-4 pm. In some implementations, the patient has each child scheduled with the same providers. In some other implementations, the patient has each child scheduled with all different providers but during the same time range. In some implementations, the preference data is represented by a checkbox selection in a user interface of the client device representing whether the appointment slots should overlap or be contiguous.

The appointment management system determines an aggregate appointment duration and provider type data based at least on the one or more procedural codes (step 204) in the request for appointment availability. The appointment management system can retrieve a mapping of procedural codes to procedural appointment durations. That is, each procedural code can map to a duration for a candidate appointment slot. For example, the duration of a procedure having a procedural code of D1 can be for 40 minutes. The duration can be a preset value provided by the practice. In some implementations, the duration is automatically calculated from a history of appointments having the particular procedural code. For example, the duration can be an average or median of the durations of appointments associated with the procedural code stored in the appointment management system. With multiple procedural codes, the appointment management system can determine an aggregate appointment duration by summing up the appointment durations for each procedural code in the request.

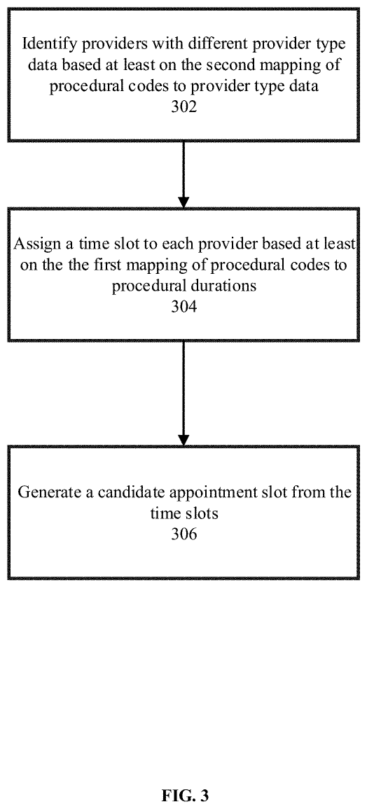

The appointment management system can also determine provider type data from a mapping of procedural codes to provider type data. The mapping can be preset by the practice and can represent the one or more specialist or generalist providers who are needed to perform operations associated with the procedural codes. For example, the mapping may show that any assistant in addition to either specialists Dr. Black and Dr. Red at the practice can perform a provided procedural code of S1 because the procedural code requires one assistant and one specialist matching the provider type for that procedural code. The provider type data can be retrieved from provider data, e.g., provider data 112 referenced in FIG. 1. This will be described further below in reference to FIG. 3.

The appointment management system identifies appointment constraints based at least on the request for appointment availability and procedural codes, the aggregate appointment duration, and the provider type data (step 206). Each appointment constraint can represent a condition that needs to be satisfied, i.e., a hard constraint, or optimally is satisfied, i.e, a soft constraint, when generating candidate appointment slots. Constraints can be set as a hard constraint or a soft constraint by operators of the practice stored in the appointment management system.

The data included in the request for appointment availability can be represented as appointment constraints. That is, the aggregate duration, provider types, and patient preference data can be an initial set of appointment constraints that must be satisfied by any candidate appointment slots. By way of illustration, one appointment constraint can be that the candidate appointment slots have a particular duration, e.g., 30 minutes, or that the candidate appointment slots include times where a particular provider has calendar availability. Each procedural code can be associated with patient data, provider data, and office data, and the appointment management system can generate constraints from each of these datasets as stored in the practice management data. This will be described further below in reference to FIG. 5.

In some implementations, additional appointment constraints include room availability based on calendar availability data for the practice, inventory availability, calendar availability for the providers, office opening hours, preferred office operating hours, provider working hours, provider preferred working hours, provider holidays, provider to procedure preference data, provider to room preference data, or provider double-booking preferences. Provider preferred working hours represent a preference during which procedures should be scheduled. Provider to procedure preference data represents a preference that providers have to perform or not to perform certain procedures. For example, one provider can prefer to perform cleanings, another provider can prefer to do anything but perform cleanings, and these preferences can be stored in the appointment management system. Provider to room preference data represents a preference that providers have to certain rooms in the practice. Provider double-booking preference represents a preference for a provider to allow for double-booking of appointments for his or her patients. Thus, the appointment management system can book up to two appointments per time block for the provider with this preference. These constraints can be established by the practice and stored in the practice management data, e.g., the practice management data 108 referenced in FIG. 1.

Since the original request for appointment availability includes one or more procedural codes, the appointment management system can also consider the additional constraints associated with the procedural code when generating candidate appointment slots such as whether inventory for the procedure will be available, which providers can perform the procedure, the provider to room preference data, provider double-booking preferences, or other constraints described above.

In some implementations, these constraints are learned using machine-learning techniques based on historical appointment data from the practice management data. In particular, the appointment management system can train a machine learning model to infer constraints given a procedural code. The model can be trained on training data based on historical appointment data that includes data labeling an appointment slot, procedural codes, duration, provider data, patient data, and office data, e.g., room in which the appointment occurred. The appointment management system can then infer constraints from a given input procedural code.

The appointment management system applies the appointment constraints to calendar availability data of a plurality of providers to generate candidate appointment slots (step 208). The candidate appointment slots need to satisfy all hard constraints and are prioritized according to the number of satisfied soft constraints. The calendar availability data includes data indicating calendar availability of the practice, of each provider, and of the patient and, if applicable, patient family members. In some implementations, the appointment management system generates candidate appointment slots by identifying time ranges in calendar availability data where at least one provider is available or is unoccupied. The appointment management system can further identify candidate appointment slots from calendar availability data that satisfy all conditions outlined by the identified appointment constraints. Example candidate appointment slots are described further below in reference to FIG. 5.

FIG. 3 is a flow chart of an example process of generating candidate appointment slots based on provider data by the appointment management system. In some implementations, each candidate appointment slot includes time slots for multiple providers of different provider types. For example, a procedural code for a cleaning can be used to generate a candidate appointment slot including a time slot for a dentist and a time slot for a hygienist.

The appointment management system identifies providers with different provider type data based at least on a mapping of procedural codes to provider type data. The provider type data can be a category or specialty of a given provider. Each procedural code can map to multiple categories or specialties. That is, each procedure can require different providers to perform the procedure. The mapping can also specify an ordering of the providers for the given procedural code. By way of illustration, a cleaning procedure can have a code that maps to two provider types: a hygienist and a dentist with the hygienist being ordered first and the dentist ordered second. The appointment management system can identify the office hygienists and the dentists from the practice management data.

The appointment management system assigns a time slot to each provider based at least on the mapping of procedural codes to procedural durations (step 304). The time slots can be identified by applying the appointment constraints to calendar availability data as described above in reference to FIG. 2. Following the illustration above, a procedural code referencing a cleaning can take 30 minutes. The appointment management system assigns an available hygienist and an available dentist to a candidate time slot. In particular, the appointment management system can assign the available hygienist to the first part of the candidate time slot and assign the dentist to the second part of the candidate time slot due to the mapping from the procedural code to provider type data.

The appointment management system generates candidate appointment slots from the associated time slots (step 306). Upon acceptance of the candidate appointment slot, the appointment management system marks the respective time slot in the candidate appointment slot for each provider as busy in the calendar availability data.

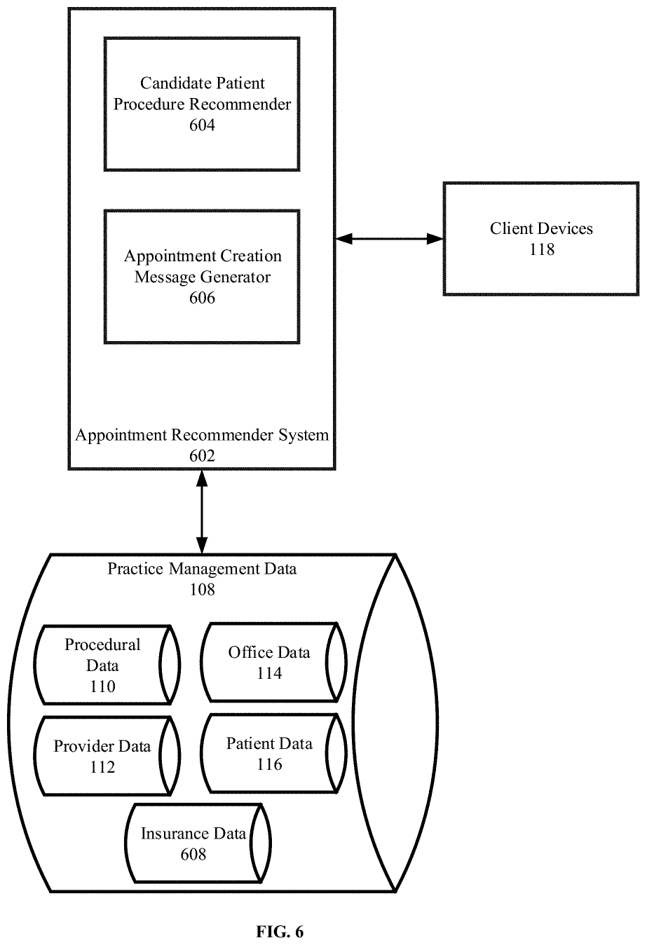

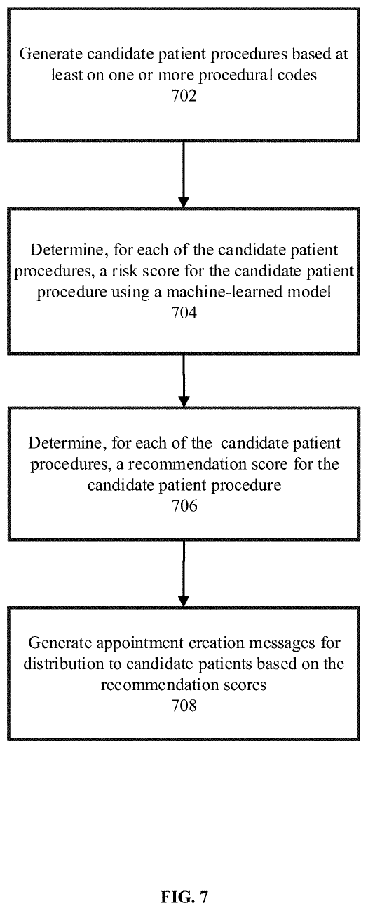

In some implementations, the appointment management system generates data indicating lost revenue from a mapping of procedural codes to revenue. The appointment management system can analyze open calendar availability in any given time period and identify time ranges during which appointments were not scheduled. The appointment management system can then calculate a corresponding lost revenue estimate for the open calendar space based on charts of patients who have pending procedures to be scheduled and the associated procedural codes for the pending procedures. The revenues can be obtained from mappings of procedural codes to billable cost. Calculating revenue estimates are described further below in reference to FIGS. 6-7.

FIG. 4 is an illustration of an example user interface showing data representing procedural codes and associated appointment durations. Procedural Codes 402 show example procedural codes D1, D2, D3, D4, and D5 which are mapped to procedure names 404 as well as durations 406 and provider types. Durations 406 can be procedural or appointment durations established by the practice or inferred as described above. Each letter in the duration can represent ten minute chunks in a calendar. For example, a duration of ADDD represents an assistant, e.g., a first provider type, should be scheduled for the first ten minutes for this procedure and the dentist, e.g., a second provider type, should be scheduled for the latter thirty minutes for this procedure. Similarly, a duration of ADA represents an assistant should be scheduled for the first and last ten minutes of the thirty minute appointment while the dentist should be scheduled for the middle ten minutes of the thirty minute appointment.

In some implementations, the candidate appointment slots include, for each time partition during the appointment slot, an associated provider having an open calendar during that time. For example, for a procedural code D1, one candidate appointment slot can specify a time from 9:00 AM to 9:40 AM with John the Hygienist booked from 9:00 AM to 9:10 AM and Dr. Red booked from 9:10 AM to 9:40 AM while another candidate appointment slot can specify a time from 1:00 PM to 1:40 PM with John the Hygienist booked from 1:00 PM to 1:10 PM and Dr. Red booked from 1:10 PM to 1:40 PM.

FIG. 5 is an illustration of an example user interface showing generation of candidate appointment slots. The user interface provides, to the appointment management system, one set of appointment constraints when generating candidate appointment slots. An operator of the user interface, e.g., a patient or an operator of the practice, can select a type of procedure 502 for scheduling. Each procedure listed in the user interface can map to one or more procedural codes to be sent in a request for appointment availability. The operator of the user interface can select preferences 504 that will serve as appointment constraints to be considered by the appointment management system such as the days, times, or providers for the particular procedure. Upon selection of the preferences and procedures, the user interface can send the request for appointment availability to the appointment management system, which will then consider additional appointment constraints identified by an appointment constraint generator, e.g., the appointment constraint identifier 106 reference in FIG. 1. These additional constraints can include office opening hours, provider calendar availability, provider preference, and other constraints described above. The appointment management generator can then provide candidate appointment slots to the user interface for display.

In this example, while the user interface may display one provider, e.g., Dr. Black, as being available for the procedure from 9 AM-10 AM, should the appointment be set, the candidate time slot can include multiple time slots assigned to different providers. That is, the appointment management system can assign the 9 AM-9:10 AM time slot to a hygienist for the procedure and 9:10 AM-LOAM to Dr. Black. The hygienist would have open calendar availability outside of 9:00 AM-9:10 AM and Dr. Black would have open calendar availability outside of 9:10 AM-10 AM.

FIG. 6 is a schematic illustration of an example appointment recommendation architecture. The appointment recommendation architecture includes an appointment recommender system 602 that interfaces with practice management data 108 and client devices 118, e.g., as described in reference to FIG. 1.

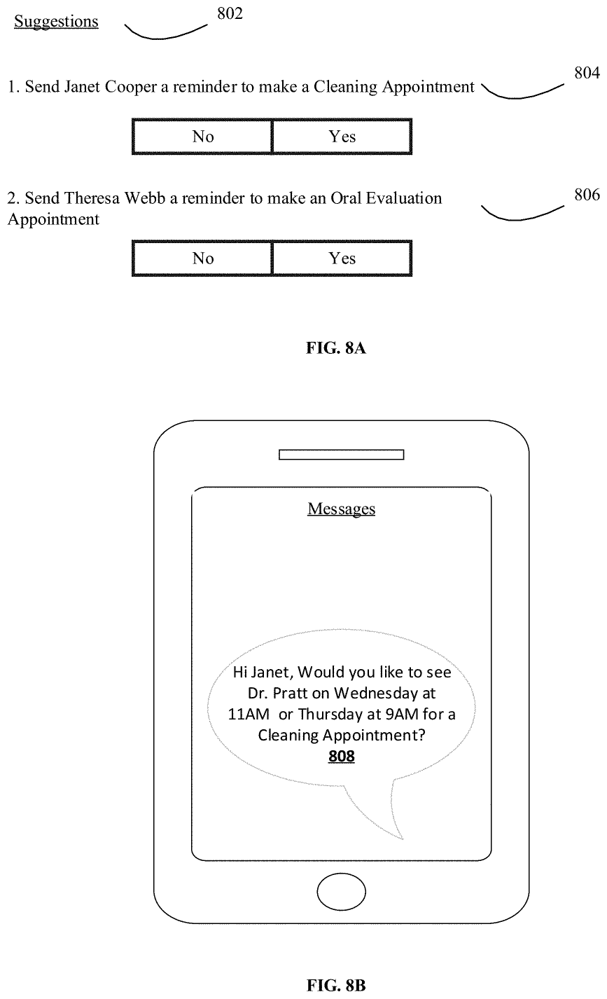

The appointment recommendation system 602 includes a candidate patient procedure recommender 604 and an appointment creation message generator 606. The candidate patient procedure recommender 604 processes practice management data 108 to generate recommendations for patient procedures that are provided to the client devices 118. The appointment creation message generator 606 processes practice management data 108 and the generated recommendations to create messages that will be sent to patients of the practice, e.g, through client devices 118. Generating recommendations for patient procedures will be described further below in reference to FIGS. 7-8.

In some implementations, the practice management data 108 includes insurance data 608. Insurance data includes information about insurance providers such as name, contact information, mappings from procedural codes to billable cost, covered procedures, and claims data including submitted, rejected, appealed, and corrected claim filings. Claim data can also include procedural codes mapped to claim filings, supporting documentation such as photos or radiographs, as well as narratives for performance of procedures.

FIG. 7 is a flow chart of an example process of generating appointment creation messages based on recommendation scores. The process can be executed by an appointment recommendation system, e.g., using an appointment recommendation system 602 referenced in FIG. 6.

The appointment recommendation system generates candidate patient procedures based at least on one or more procedural codes (step 702). The appointment recommendation system can access charts for all patients from the practice management data to identify future procedures for scheduling. Each future procedure is associated with a procedural code. For example, the appointment recommendation system can automatically assign a cleaning to each patient every six months, and the cleaning is added to the patient's chart and stored in the patient data. The appointment recommendation system then can identify all future procedures over a given timeframe as candidate patient procedures. In some implementations, the appointment recommendation system filters candidate patient procedures by procedural codes provided by the practice.

The appointment recommendation system determines, for each candidate patient procedure, a risk score for the candidate patient procedure (step 704). The risk score can be generated from a machine-learning model trained on data including one or more procedure types, patient information, and frequency scores of procedural incidents for each procedure type, and severity scores of procedural incidents for each procedure type. In some implementations, the data includes the duration of the procedures. The data can be historical data stored in the practice management data. In some implementations, a subset of the data listed above is used to train the machine-learning model. Procedure types can be represented by procedural codes, procedure names, or other procedural identifiers. Patient information can include properties like age and gender. Frequency and severity scores of procedural incidents can be sourced from the patient data and anonymized for use in the machine learning model. For example, a procedural incident can be labeled to have occurred if a dentist adds a note that a filling in a molar tooth had to be replaced as a result of a procedure. In some implementations, severity scores are labeled by experts training the machine-learning model. In some other implementations, severity scores are assigned automatically by processing notes associated for each procedure using a sentiment classifier trained on unstructured data. By way of illustration, an accident occurring because of an error from a dentist can have a higher severity score than an incident where a patient requests for a different type of cleaning gel. The risk score in the training data can be labeled by experts or calculated from a function based on the frequency or severity scores. The appointment recommendation system can train the machine learning model using supervised or unsupervised learning techniques. The machine learning model can output a risk score given an input procedural code.

In some implementations, determining the risk score is an optional step before determining the recommendation score.

The appointment recommendation system determines, for each candidate patient procedure, a recommendation score for the candidate patient procedure (step 706). The recommendation score can be based at least on insurance provider data for the patient, a mapping of the candidate patient procedure to revenue per procedure, and the risk score for the candidate patient procedure. The appointment recommendation system can identify insurance provider data associated with the procedure using the practice management data. The insurance provider data can include a mapping of covered procedures to costs paid per covered procedure. For example, insurance provider data can include data indicating Insurance A will pay $X for procedural code D1, e.g., an oral evaluation. Insurance provider data can also include a frequency of claims that were rejected or changed as well as a preference score established by the practice. The revenue per procedure can be established by the practice or can be learned and dynamically adjusted over time from historical revenue data stored in the practice management data. The recommendation score can be determined by combining a weighted revenue score and a weighted risk score.

In some implementations, the recommendation score is learned from a separate machine learning model trained on data including insurance provider data for the patient, a mapping of the candidate patient procedure to revenue per procedure, and the risk score for the candidate patient procedure. The recommendation score can be labeled by experts or operators of the practice. In some implementations, the machine learning model can be combined or replaced with the model described above in step 704. For example, the machine learning model can be trained on data including procedure types, patient information, and frequency scores of procedural incidents for each procedure type, severity scores of procedural incidents for each procedure type, insurance provider data for the patient, a mapping of the candidate patient procedure to revenue per procedure, and a risk score, all of which can be used to infer a recommendation score. While the models described above reference training on multiple types of data, a subset of the data can be used to train the model.

In some implementations, the data affecting the outcome of the recommendation score is weighted according to a preference of the practice. For example, the practice can prioritize recommendations having higher revenue amounts, lower risk scores, or low severity scores. In some implementations, the data is weighted to prioritize procedural preferences of the providers.

The appointment recommendation system generates one or more appointment creation messages for distribution to a plurality of candidate patients based on the recommendation scores (step 708).

In some implementations, the appointment recommendation system generates one or more appointment creation suggestions based on the recommendation scores. The suggestions can be sent to client devices for approval. For example, the appointment recommendation system can generate top suggestions sorted by recommendation score to be provided to operators, e.g., health administrators, of the appointment recommendation system. The operators can approve the suggestions and send approvals to the appointment recommendation system. Upon receiving the approval of the suggestions, the appointment recommendation system can generate appointment creation messages and send them to other client devices associated with the appointments specified in the creation messages, e.g., devices of the patients of the practice.

In some implementations, appointment creation messages include candidate appointment slots generated from the processes described above in reference to FIG. 2. For example, the appointment recommendation system identifies appointment constraints based on the candidate patient procedures, which were recommended by the appointment recommendation system. The appointment constraints can, for example, include preference data from providers of the practice. The appointment recommendation system can then apply the appointment constraints to calendar availability data to generate candidate appointment slots, which will be included in the appointment creation messages. Appointment creation messages will be described further below in reference to FIGS. 8A-8B.

FIG. 8A is an illustration of an example user interface showing a generated appointment creation message. The generated appointment creation messages can be displayed on a user interface on a client device, e.g., an operator of the practice can see the suggestions on a client device. The user interface shows suggestions 802 which, when triggered, can send approval of the suggestion to the appointment recommendation system. For example, the appointment recommendation system can generate a suggestion 804 to send Janet Cooper a reminder to make a cleaning appointment. The appointment recommendation system can identify that Janet Cooper is charted to receive an additional cleaning at some point in the future, that among the many other potential procedures to be scheduled, this procedure has a low risk score, low severity score, high provider preference, or high revenue potential and thus suggests an appointment creation message for this procedure instead of other potential procedures. Once the appointment recommendation system receives an approval of the suggestion, the appointment recommendation system can generate an appointment creation message for distribution.

FIG. 8B is an illustration showing the appointment creation message being distributed to a client device. The appointment recommendation system identifies contact information from patient data associated with a given recommended procedure and sends the patient a message 808. The message 808 includes information about the recommended procedure including the procedure type and provider, one or more candidate time slots, and can be displayed on a client device.

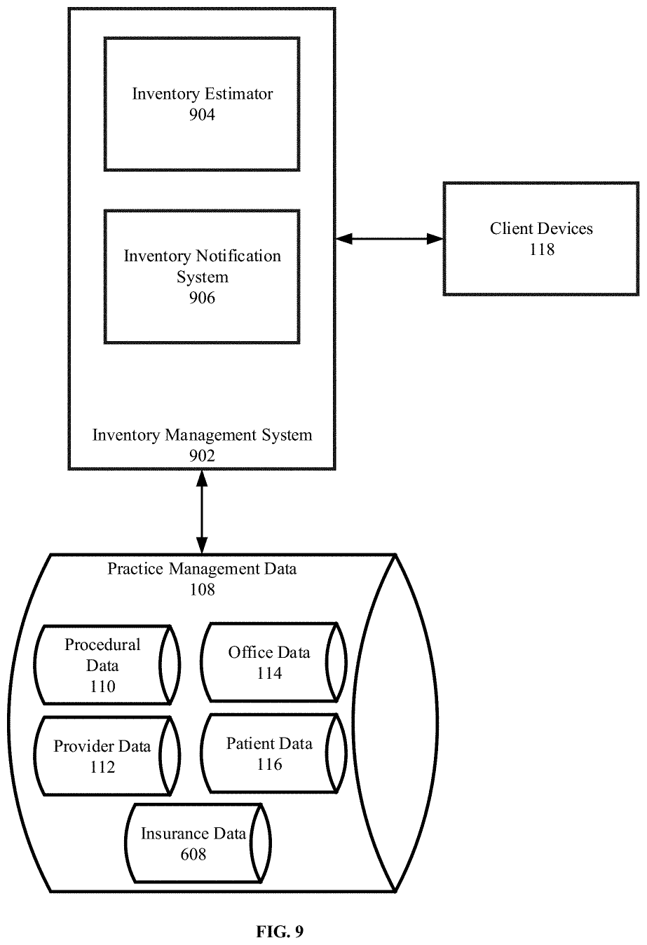

FIG. 9 is a schematic illustration of an example inventory management architecture. The inventory management architecture includes an inventory management system 902 that interfaces with practice management data 108 and client devices 118, e.g., as described in reference to FIG. 1.

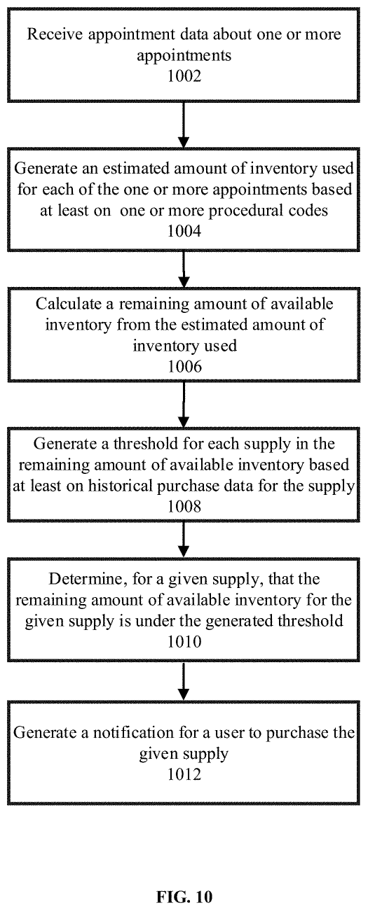

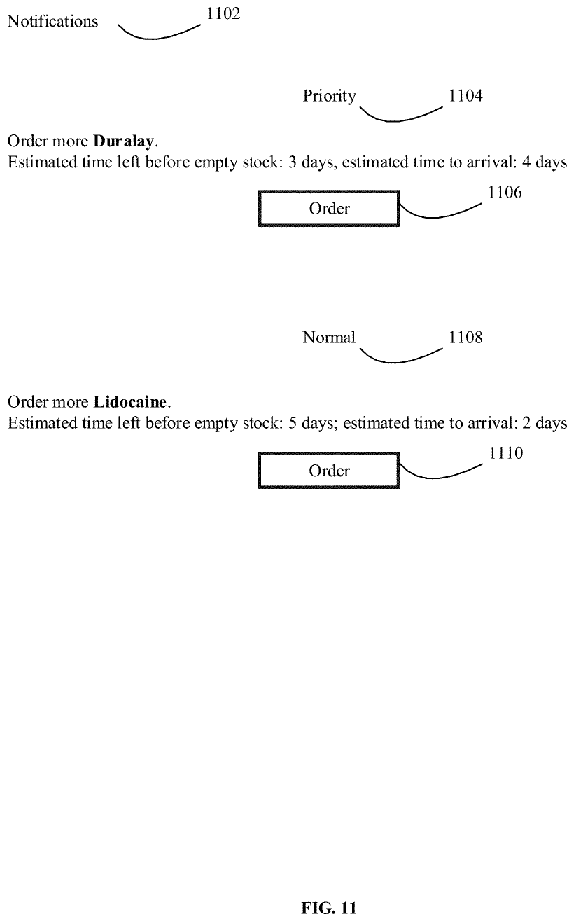

The inventory management system 902 includes an inventory estimator 904 and an inventory notification system 906. The inventory estimator 904 processes practice management data 108 to estimate available inventory for the practice. Inventory can include supplies such as amalgam, lidocaine topical, hurricaine spray, topical anesthetic, monoject needles, protector needle sheaths, or other medical or dental inventory. The inventory management system 902 can track and store the amount of supply owned by the practice. The inventory notification system 906 processes practice management data 108 to generate notifications to order more inventory. The notifications can be sent to client devices 118 owned by operators of the practice. Estimating inventory for the practice and notification sending will be described further below in reference to FIGS. 10-11.

FIG. 10 is a flow chart of generating a notification for a user to purchase supply for more inventory. The process can be executed by an inventory management system, e.g., using an inventory management system 902 referenced in FIG. 9.

The inventory management system receives appointment data about one or more appointments (step 1002). The appointment data can be accessed in the practice management data, e.g., practice management data 108 referenced in FIG. 9. The appointment data includes one or more procedural codes for each of the one or more appointments, associated provider data, office data, other procedural data and insurance data.

The inventory management system generates an estimated amount of inventory used for each of the one or more appointments based at least on the one or more procedural codes (step 1004). The inventory management system can use a mapping from procedural codes to inventory used to estimate the amount of inventory used per appointment. The mapping can be preset by the practice or can be learned from historical usage of the practice. That is, operators of the practice can manually input, into the inventory management system, an amount of inventory used for each procedural code at each appointment, and the inventory management system can calculate an average or median for use in the mapping. In some implementations, the inventory management system calculates an amount of inventory used per procedural code based on the amounts of inventory purchased through the inventory management system and the procedures performed between inventory purchases. If an appointment has more than one procedural code, the inventory management system can add, for each procedural code, the amount of inventory used to the estimate according to the mapping.

The inventory management system calculates a remaining amount of available inventory from the estimated amount of inventory used (step 1006). The inventory management system tracks, e.g., in the practice management data, the amount of available inventory for the practice. The remaining amount of available inventory can be calculated by subtracting the estimated amount of inventory used from the amount of available inventory for the practice.

The inventory management system generates a threshold for each supply in the remaining amount of available inventory based at least on historical purchase data for the supply (step 1008). In some implementations, the historical purchase data specifies a duration from order to arrival. For example, historical purchase data for Duralay can include an amount purchased, a purchase order date, a cost amount, and an arrival date. The historical purchase data can inform how long shipment of each supply would take. Based on the duration, the inventory management system can generate a threshold supply amount that, when the supply is under the threshold, an order for the supply should be placed so the supply can reach the practice in time, which would allow procedures with that procedural code to not be limited by supply. The threshold is thus also based on the rate of procedures utilizing the supply. In some implementations, the threshold takes into account a buffer notification time which the practice can use to consider whether to make a purchase order for the inventory. An example will be described further below in reference to FIG. 11.

In some implementations, the inventory management system automatically places an order for the inventory that goes under the threshold without input from an operator of the practice. In some other implementations, the threshold is inferred from a machine learning model trained on data including inventory data, date data, historical appointment data, and the historical purchase data.

The inventory management system determines, for a given supply, that the remaining amount of available inventory for the given supply is under the generated threshold (step 1010). Once enough procedures with procedural codes using the given supply have been performed, have been scheduled to be performed, the inventory management system can determine the threshold has been triggered, e.g., the amount of available inventory is under the threshold.

In some implementations, the inventory management system determines that the threshold has been satisfied when inventory is being consumed at a particular rate. The rate of consumption can be calculated from an inventory estimate used by a number of procedures over a given period of time. For example, despite the number of procedures scheduled to be performed at the practice, if a rate of consumption reaches the threshold, the inventory management system can determine a notification to purchase more inventory should be sent to avoid running out of supply.

The inventory management system generates a notification for a user to purchase the given supply (step 1012). In some implementations, generating the notification is in response to determining the threshold has been triggered.

In some implementations, an operator can track inventory and provide the updated inventory amount to the inventory management system. The inventory management system can adjust the remaining amount of inventory for the practice based at least on the updated inventory amount.

In some implementations, the inventory management system generates a priority for the notification. Because some procedures are performed at different frequencies than others, the inventory management system can prioritize the supplies used by the most frequent procedures to minimize loss of revenue or minimize risk if the procedures were canceled due to lack of supply. The revenue can be calculated according to insurance data mappings from procedural codes to billable amounts. The amount of revenue can be represented by a revenue score and risk per procedure can be represented by a risk score determined by the practice. The revenue and risk scores can be established by the practice. In some implementations, the priority for the notification is determined by how much buffer time has elapsed. The priority of the notification can be conveyed to an operator of the practice through a client device, which will be described further below in reference to FIG. 11.

FIG. 11 is an illustration of an example user interface showing a notification to purchase inventory based on available inventory. The user interface can be displayed on a client device, e.g., client devices 118 referenced in FIG. 9.

The inventory management system can generate the notifications 1102 that are shown on the display. The notifications can be split according to priorities, e.g., high priority 1104 and normal priority 1108. The notification can display an estimated time left before there is no more available inventory for procedures with that procedural code as well as an estimated time for the inventory to arrive at the practice. The time left can be calculated according to procedures scheduled with the practice or a rate of inventory consumption, as described above in reference to FIG. 10. The user interface can display buttons labeled “Order” 1106 and 1110 which when triggered will cause the inventory management system to place an order with the manufacturer of the supply for the practice.

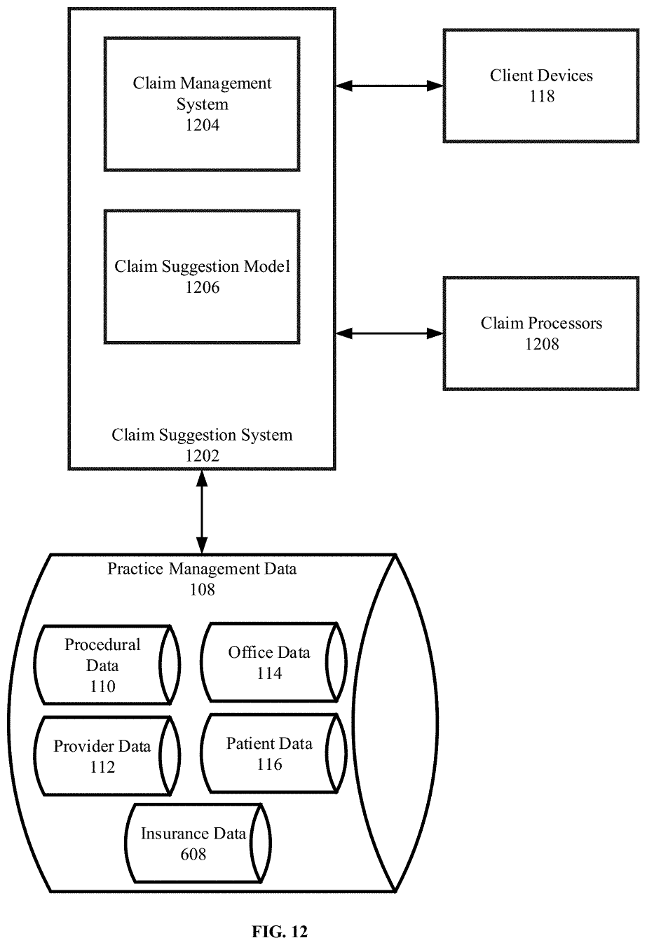

FIG. 12 is a schematic illustration of an example claim filing management architecture. The claim filing management architecture includes a claim suggestion system 1202 that interfaces with practice management data 108 and client devices 118, e.g., as described in reference to FIG. 1.

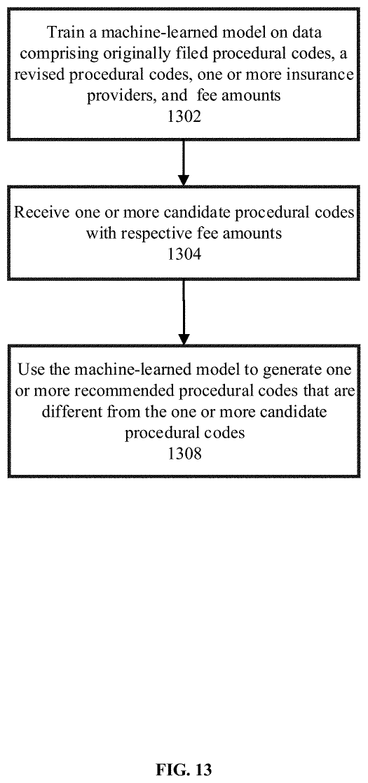

The claim suggestion system 1202 includes a claim management system 1204 and a claim suggestion model 1206. The claim management system 1204 processes practice management data 108 to track, submit, and edit claim filings to be sent to claim processors 1208, which are systems operated by insurance providers. The claim processors 1208 can receive the claim filings and send back approval or rejections of the claim filings for processing by the claim management system 1204. The claim management system 1204 uses the claim suggestion model 1206 to generate recommendations for submission to the claim processors 1208. Generating claim suggestions will be described further below in reference to FIGS. 13-15.

FIG. 13 is a flow chart of an example process of generating recommended procedural codes for claim filings. The process can be executed by a claim suggestion system, e.g., using a claim suggestion system 1202 referenced in FIG. 12.

The claim suggestion system trains a machine-learning model, e.g., claim suggestion model 1206 referenced in FIG. 12. The machine-learning model can be trained using data including originally filed procedural codes, revised procedural codes, one or more insurance providers, fee amounts, descriptions and notes for the performed procedures, and associated approval statuses. For example, each data point can represent a claim filing including one or more procedural codes, associated fee amounts for each procedural code, an insurance provider handling the claim filing, and whether the claim was approved or denied, and revised procedural codes provided by the insurance provider, if applicable. In some implementations, the data can also include date data associated with the originally filed procedural codes or the claim filing. In some implementations, the notes are converted to an embedding for training and inference. The training data represents historical claim filing data for the practice, and in some implementations, are combined with claim filing data from other practices.

This machine-learning model and other machine-learning models described herein can be trained using machine learning algorithms including linear or logistic regressions, K-means clustering, random forest, or support vector machines. This machine-learning model can take, as input, procedural codes, an insurance provider, and optionally, a date, and the machine-learning model outputs revised procedural codes if applicable. In this way, the machine-learning model can be used by the practice to submit more accurate claim filings to claim processors of the insurance provider.

The claim suggestion system receives one or more candidate procedural codes with respective fee amounts. This can be provided by an operator of the claim suggestion system, e.g., an administrator of the practice, intending to submit a claim to claim processors of an insurance provider for payment by the insurance provider.

In some implementations, the machine-learning model determines that the candidate procedural codes do not need to be adjusted before submission to the claim processors.

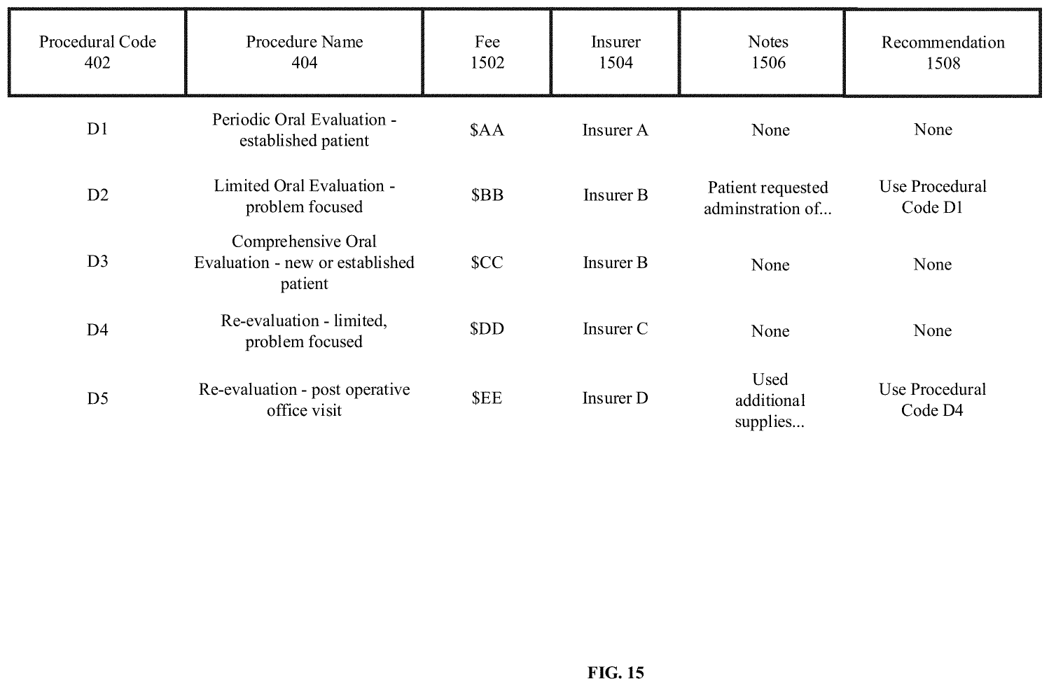

In some other implementations, the claim suggestion system uses the machine-learning model to generate one or more recommended procedural codes that are different from the one or more candidate procedural codes. The claim suggestion system can provide the one or more candidate procedural codes to the client devices through a user interface, which will be described further below in reference to FIG. 15.

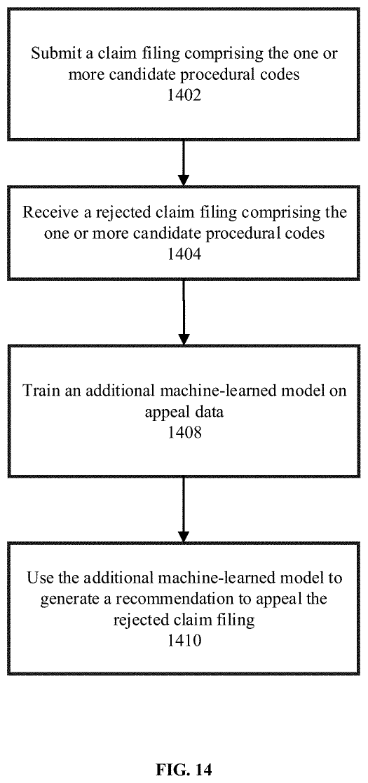

FIG. 14 is a flow chart of an example process of generating a recommendation to appeal rejected claims. The process can be executed by a claim suggestion system, e.g., using a claim suggestion system 1202 referenced in FIG. 12.

The claim suggestion system submits claim filing data including the one or more candidate procedural codes. The claim data can be submitted to a claim processor. The claim data can also include a date, a fee amount for which an insurance provider should pay to the practice, and descriptions of the one or more performed procedures. For context, after the claim suggestion system submits a claim to a claim processor, the claim processor can reject the claim at its discretion.

The claim suggestion system receives rejected claim filing data including the one or more candidate procedural codes. In some implementations, the rejected claim data includes a potential revised procedural code for resubmission or notes indicating reasoning for rejection. The rejected claim data can be stored by the claim suggestion system and used to train the machine-learning model used to infer revised procedural codes.

In particular, the claim suggestion system can train an additional machine-learning model on appeal data including insurance provider data, historical claim filing data including first fee data and first procedural code data, historical claim rejection data provided by a claim processor including second fee data and second procedural code data, and historical claim appeal data including final fee data and final procedural code data. The final fee data and final procedural code data can be used in a revised claim filing to a claim processor for approval by the insurance provider. The appeal data can also include labeled data indicating a claim filing should be appealed if any revised claim filing resulted in approval by the claim processor. Thus, based on the training data, the additional machine-learning model can take, as input, rejected claim data and output a recommendation to submit an appeal to the claim processor.

The claim suggestion system uses the additional machine-learning model to generate a recommendation to appeal the rejected claim filing. If the appeal is approved, the claim insurance provider can provide payment to the practice in the amount of the original submission of the claim.

FIG. 15 is an illustration of an example user interface showing recommended procedural codes for claim filings. The user interface shows procedural codes 402 with associated procedure names 404, associated fees 1502, insurers 1504, notes 1506, and recommendations 1508. The notes 1506 can be provided by a provider, e.g., dentist, from the practice. Each row in the user interface can represent a claim filing to a claim processor. The recommendations 1506 can be generated by a claim suggestion system, e.g., claim suggestion system 1202 referenced in FIG. 12. The claim suggestion system 1202 can provide a recommendation using a claim suggestion model to change procedural codes for a given procedure based on the fee amount, insurer, notes from the provider, and original procedural code. For example, for a claim filing with procedural code D2 and associated metadata, the claim suggestion system can recommend to use procedural code D1 instead of D2 for the claim filing based on the insurer, fee, procedural code, and notes of the claim filing. The operator can then optionally choose to amend the claim filing prior to submission to a claim processor to maximize approval rates for claim filings. The claim suggestion system 1202 can then replace the candidate procedural codes in the original filing with the recommended procedural codes from the claim suggestion model and submit a revised claim filing to the claim processors.

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on a non-transitory computer storage medium for execution by, or to control the operation of, data processing apparatus. Alternatively or in addition, the program instructions can be encoded on an artificially generated propagated signal, e.g., a machine generated electrical, optical, or electromagnetic signal, that is generated to encode information for transmission to suitable receiver apparatus for execution by a data processing apparatus. A computer storage medium can be, or be included in, a computer readable storage device, a computer readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language resource), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending resources to and receiving resources from a device that is used by the user; for example, by sending web pages to a web browser on a user's client device in response to requests received from the web browser.

Embodiments of the subject matter described in this specification can be implemented in a computing system that includes a backend component, e.g., as a data server, or that includes a middleware component, e.g., an application server, or that includes a frontend component, e.g., a client computer having a graphical user interface or a Web browser through which a user can interact with an implementation of the subject matter described in this specification, or any combination of one or more such backend, middleware, or frontend components.

The computing system can include clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

Thus, particular embodiments of the subject matter have been described.

1. A method comprising, by one or more computing devices:
receiving appointment data about one or more appointments, wherein the appointment data comprises one or more procedural codes for each of the one or more appointments;
generating an estimated amount of inventory used for each of the one or more appointments based at least on the one or more procedural codes;
calculating a remaining amount of available inventory from the estimated amount of inventory used;
generating a threshold for each supply in the remaining amount of available inventory based at least on historical purchase data for the supply, wherein the historical purchase data comprises a duration from order to arrival;
determining, for a given supply, that the remaining amount of available inventory for the given supply is under the generated threshold; and
in response to the determining, generating a notification for a user to purchase the given supply.

2. The method of claim 1, wherein generating the threshold for each supply comprises:
training a machine learning model on data comprising inventory data, date data, historical appointment data, and the historical purchase data;
inferring, using the trained machine learning model, the generated threshold.

3. The method of claim 1, wherein generating the threshold is based at least on a rate of consumption of each supply.

4. The method of claim 1, further comprising:
receiving an updated inventory amount; and
adjusting the remaining amount based at least on the updated inventory amount.

5. The method of claim 1, further comprising assigning the notification a priority based on at least a frequency of procedural codes in the appointment data.

6. The method of claim 1, further comprising assigning the notification a priority based on at least revenue score or a risk score.

7. A system comprising:
a processor; and
computer-readable medium coupled to the processor and having instructions stored thereon, which, when executed by the processor, cause the processor to perform operations comprising:
receiving appointment data about one or more appointments, wherein the appointment data comprises one or more procedural codes for each of the one or more appointments;
generating an estimated amount of inventory used for each of the one or more appointments based at least on the one or more procedural codes;
calculating a remaining amount of available inventory from the estimated amount of inventory used;
generating a threshold for each supply in the remaining amount of available inventory based at least on historical purchase data for the supply, wherein the historical purchase data comprises a duration from order to arrival;
determining, for a given supply, that the remaining amount of available inventory for the given supply is under the generated threshold; and
in response to the determining, generating a notification for a user to purchase the given supply.

8. The system of claim 7, wherein generating the threshold for each supply comprises:
training a machine learning model on data comprising inventory data, date data, historical appointment data, and the historical purchase data;
inferring, using the trained machine learning model, the generated threshold.

9. The system of claim 7, wherein generating the threshold is based at least on a rate of consumption of each supply.

10. The system of claim 7, further comprising:
receiving an updated inventory amount; and
adjusting the remaining amount based at least on the updated inventory amount.

11. The system of claim 7, further comprising assigning the notification a priority based on at least a frequency of procedural codes in the appointment data.

12. The system of claim 7, further comprising assigning the notification a priority based on at least revenue score or a risk score.

13. A computer-readable medium having instructions stored thereon, which, when executed by one or more computers, cause the one or more computers to perform operations for:
receiving appointment data about one or more appointments, wherein the appointment data comprises one or more procedural codes for each of the one or more appointments;
generating an estimated amount of inventory used for each of the one or more appointments based at least on the one or more procedural codes;
calculating a remaining amount of available inventory from the estimated amount of inventory used;
generating a threshold for each supply in the remaining amount of available inventory based at least on historical purchase data for the supply, wherein the historical purchase data comprises a duration from order to arrival;
determining, for a given supply, that the remaining amount of available inventory for the given supply is under the generated threshold; and
in response to the determining, generating a notification for a user to purchase the given supply.

14. The computer-readable medium of claim 13, wherein generating the threshold for each supply comprises:
training a machine learning model on data comprising inventory data, date data, historical appointment data, and the historical purchase data;
inferring, using the trained machine learning model, the generated threshold.

15. The computer-readable medium of claim 13, wherein generating the threshold is based at least on a rate of consumption of each supply.

16. The computer-readable medium of claim 13, further comprising:
receiving an updated inventory amount; and
adjusting the remaining amount based at least on the updated inventory amount.

17. The computer-readable medium of claim 13, further comprising assigning the notification a priority based on at least a frequency of procedural codes in the appointment data.

18. The computer-readable medium of claim 13, further comprising assigning the notification a priority based on at least revenue score or a risk score.
